(19) United States
(12) Patent Application Publication
(10) Pub. No.: US 2010/0184203 A1
(43) Pub. Date: Jul. 22, 2010

(54) STRAW EQUIPPED WITH A PLUG COMPRISING A FILTERING ELEMENT

(75) Inventors: Eric Schmitt, Villaines-La-Juhel (FR); Alain Legal, Rochefort En Terre (FR)

Correspondence Address: BROWDY AND NEIMARK, P.L.L.C.;624 NINTH STREET, NW, SUITE 300, WASHINGTON, DC, 20001-5303, US

(73) Assignee: IMV Technologies, L'Aigle (FR)

(21) Appl. No.: 12/664,553

(22) PCT Filed: Jun. 12, 2008
(86) PCT No.: PCT/FR08/00803

§ 371 Date: Dec. 14, 2009

(30) Foreign Application Priority Data
Jun. 12, 2007 (FR) 0755712

(51) Int. Cl. C12M 1/24

(52) U.S. Cl. 435/307.1

(57) ABSTRACT

This is a straw that comprises a tube (11) equipped with a plug (12) comprising a filtering element that is apart of the end (16) of the plug facing towards the end (15) of the tube furthest from the plug. Such a straw is used for storing a predetermined dose of a liquid-based substance, in particular a biological substance.

The invention relates to straws for storing predetermined doses of liquid-based substances, notably biological substances, for example pure or diluted animal semen or a storage medium containing embryos.

It is known that such a straw is formed conventionally by a thin tube, having an inside diameter of 1.6 to 2.5 mm, for example, and by a plug engaged in the thin tube.

In the filled state, the plug is disposed in the vicinity of a first end of the tube and the dose of substance is disposed in the straw between the plug and the second end of the tube.

To fill the straw, the first end of the tube, near the plug, is placed in communication with a vacuum source and the second end is placed in communication with a recipient containing the substance to be introduced into the straw. The air initially contained between the plug and the second end is aspirated through the plug and the substance progresses in the tube until it encounters the plug, which it cannot pass because it becomes sealed to liquids.

The filled straw is generally kept in cold storage at a temperature produced cryogenically (by liquid nitrogen), electrically (by the Peltier effect) or mechanically (by a compressor). In some cases, where the storage time is minimal, the straw is simply kept at ambient temperature.

To empty the straw, after thawing if necessary, the plug is caused to slide toward the second end of the tube, like a piston, so that the dose of substance initially contained in the straw is expelled from it through that end.

Generally speaking, straw plugs are of the tripartite type first described in French patent 995.878, corresponding to British patent 669,265, i.e. formed by two bungs made of a fibrous substance between which is a powder that is transformed in contact with a liquid into an impermeable paste or gel adhering to the wall of the tube so that the plug is sealed.

Solutions have already been proposed for limiting or even eliminating losses of substance caused by absorption by the plug.

French patent 2 753 367, to which corresponds U.S. Pat. No. 5,868,178, proposes a tripartite plug the length of the outer bung whereof is at least twice the length of the inner bung.

French patent application 2 762 210 proposes a one-piece, microporous, hydrophobic plug.

French patent applications 2 824 255 and 2 824 256, to which correspond American patent applications US 2002/0183653 and US 2002/0188222, propose adding to the plug, in addition to the powder and the fibers, non-absorbing elements, here a thermoplastic material core covered with a sheath of braided filaments and non-absorbent material dispersed in the powder.

The invention also aims to limit or even eliminate losses of substance in the plug.

To this end the invention proposes a straw for storing a predetermined dose of liquid-based substance, notably a biological substance, including a tube provided with a plug, characterized in that said plug includes a filtering element such that particles exceeding a predetermined dimensional threshold cannot penetrate into it, one end of said filtering element being part of the end of the plug facing toward the end of the tube farthest from the plug.

Thus the area of the plug reached first by the substance filling the straw is formed at least partially by the end of the filtering element.

Now, as a general rule, the most valuable material contained in the liquid-based substance, for example the spermatozoa when it is semen for use in artificial insemination, has a clearly identified dimensional threshold.

If it is dimensioned appropriately in relation to such a threshold, the plug of the straw of the invention therefore prevents this most valuable material penetrating into the plug and therefore being lost there.

It will be noted that the plug of the straw of the invention, and to be more precise the wick that this plug includes, is not necessarily sealed to liquids, in contrast to all the prior art plugs referred to above.

There are already known numerous filtering elements capable of retaining particles exceeding a predetermined dimensional threshold fixed by the gaps in or pores of the filtering elements.

To provide such a filtering element that is simple, convenient and economical whilst offering a relatively precise dimensional threshold, this filtering element preferably includes a wick of predetermined section formed of a multitude of filaments each oriented in the same general direction, each of said filaments having the same predetermined constant section over its length.

It is advantageous if a filtering element is disposed in the tube with the general direction of the wick and the general direction of the tube coinciding and with the area of the filtering element reached first by the fluid formed at least partly by the end of the wick.

In the latter wick, the identical orientation and section of all the filaments accurately control the geometrical characteristics of the spaces between the filaments seen in section.

It is in fact possible to determine from the number and the section of the filaments and the section of the wick the dimensional threshold beyond which particles encountering the wick under the conditions indicated above cannot penetrate into it and remain blocked at the end of the wick where the encounter occurs.

The invention thus offers the possibility of producing a filtering element offering excellent accuracy using simple filaments available off the shelf, for example filaments of polyester, glass fibers or carbon fibers.

According to preferred features, said wick has a length at least equal to its radius.

With such a length of the wick, and thus of the filaments that form it, the preparation of the wick and the filtering element is simple and convenient and the arrangement of the filtering element offers good stability.

According to other features preferred for reasons of simple and convenient manufacture and the quality of the results obtained:
each of said filaments has a rounded contour;
the diameter of said filaments is less than six times a prefixed diameter so that a spherical particle having a diameter greater than or equal to that prefixed diameter is blocked by said wick;
said filaments are solid;
said filaments are tubular;
the internal passages of said filaments have a diameter less than a prefixed diameter so that a spherical particle having a diameter greater than or equal to that prefixed diameter is blocked by said wick;
said wick has a retaining element around it;
said retaining element is a tube in which said wick is engaged;
said retaining element is an individual retaining sheath of said wick;
said filaments receive a surface treatment;
said surface treatment uses silicone oil;

According to features preferred for reasons of simple and convenient manufacture and the quality of results obtained:
said plug comprises exclusively said wick;
said plug includes a sheath around said wick;
said plug contains gelling powder;
said plug includes a bung between said powder and the end of said tube nearest the plug;
said bung includes one of said wicks around which is a sheath;
said bung is formed by a braid of fibrous material; and/or
said bung includes a solid core surrounded by a braid sheathing said core.

The description of the invention continues now with a detailed description of preferred embodiments given hereinafter by way of nonlimiting illustration and with reference to the appended drawings. In the latter drawings:

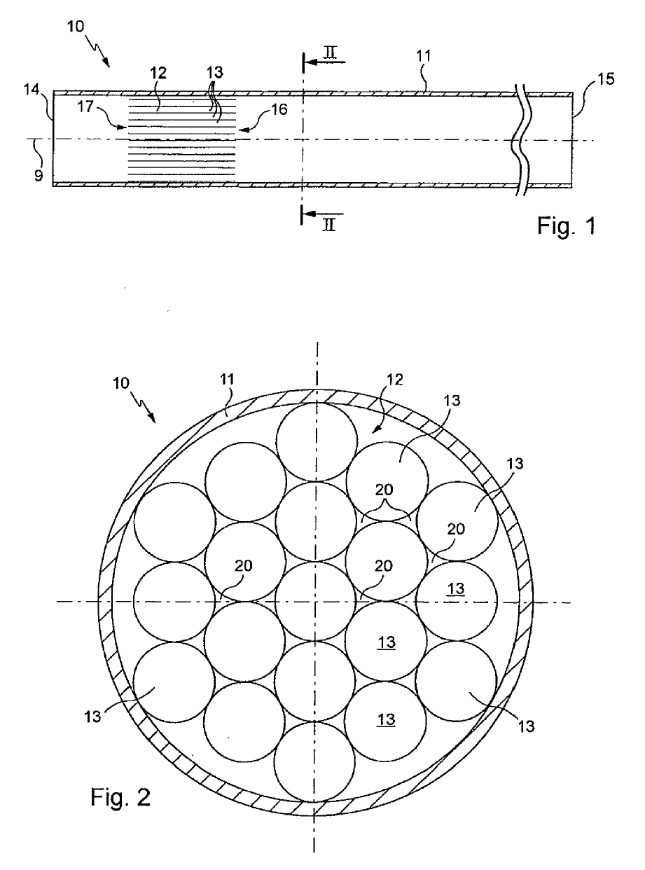

FIG. 1 is a diagrammatic view in longitudinal section of a straw including a plug formed by a filtering element;

FIG. 2 is a diagrammatic view in cross section taken along the line II-II in FIG. 1;

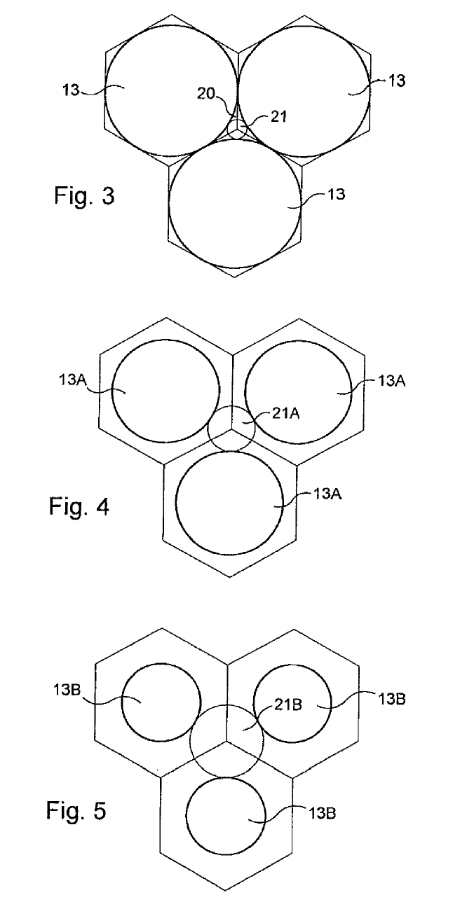

FIG. 3 is a diagrammatic view showing end-on three adjacent filaments disposed as in FIG. 2 (at the maximum rate of filling with filaments) and a geometrical construction for determining the maximum diameter that the filaments can have if the wick is required to stop spherical particles having a diameter greater than a predetermined threshold and enabling the number of filaments that constitute the wick to be determined from the maximum diameter of the filaments determined in this way and the diameter of the wick of filaments;

FIGS. 4 to 7 are similar to FIG. 3 but for decreasing rates of filling with filaments;

FIG. 8 shows in a similar way the case of a flattened rather than spherical particle;

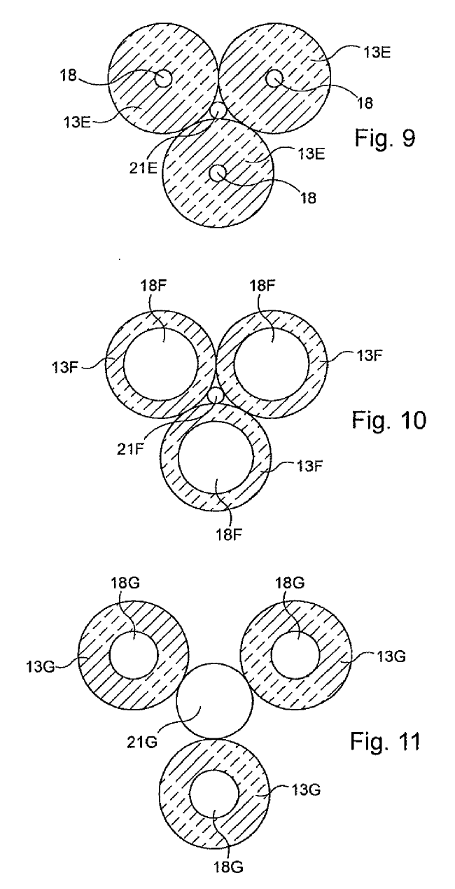

FIGS. 9 to 11 show in a similar way the case of tubular rather than solid filaments;

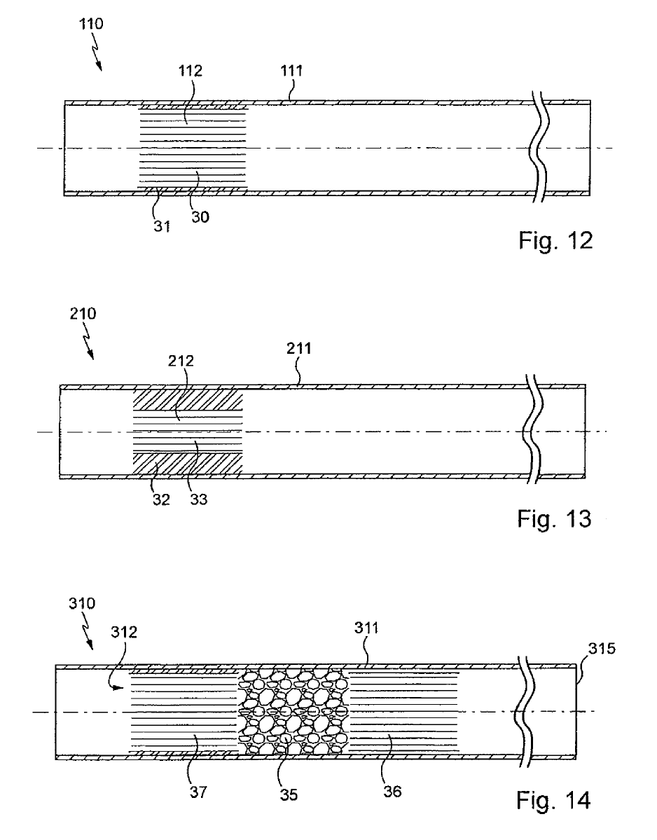

FIGS. 12 to 17 show in a similar way to FIG. 1 variants of the plug of the straw of the invention.

The straw 10 shown in FIG. 1 includes a tube 11 and a plug 12.

The tube 11 is of the standard extruded plastic material type with an inside diameter of 1.6 or 2.5 mm, for example, and a length of the order of 133 mm.

The plug 12 consists of a wick formed of a multitude of filaments 13 the quantity whereof and the characteristics whereof, notably the dimensional characteristics whereof, are predetermined.

The straw 10 is used in the standard manner.

Accordingly, in the initial state, shown in FIG. 1, the plug 12 is disposed in the vicinity of the end 14 of the tube 11 and the dose of liquid substance that is stored in the straw 10 when filled is disposed between the plug 12 and the end 15 of the tube 11 farthest from the plug 12.

To fill the straw 10, the end 14 communicates with a vacuum source and the end 15 communicates with a container containing the substance to be introduced into the straw.

The air initially contained between the plug 12 and the end 15 is aspirated through the plug and the substance progresses into the tube via the end 16 thereof facing toward the end 15 of the tube 11, i.e. the end of the plug 12 that is seen on the right in FIG. 1, until it encounters the plug 12.

As explained hereinafter, the plug 12 blocks the progress of the substance or at least of the particles of that substance exceeding a predetermined size.

If necessary, the straw is welded in the vicinity of one or both of its ends 14 and 15 and is placed in cold storage.

To empty the straw, where necessary after cutting off the welded end portions and thawing, there is caused to penetrate into the tube 11 a small rod that bears against the end 17 of the plug 12 and causes the latter to slide in the manner of a piston toward the end 15, or the corresponding end after cutting off the welded portion, which expels the dose of substance that was introduced into the straw.

There will now be more particularly explained with reference to FIGS. 2 and 3 the interaction between the liquid-based substance and the end 16 of the plug 12 when that substance encounters that end.

So as not to overcomplicate the drawings, only a particularly small number of filaments 13 have been shown.

In practice, when the tube 11 has an inside diameter of 1.6 mm, the wick that constitutes the plug 12 comprises several thousands or tens of thousands or even hundreds of thousands of filaments 13.

The filaments 13 are non-cracked and non-carded unitary elements. They must not be confused with threads, strands or braids.

A thread is produced from a number of braided or twisted filaments, for example 50 filaments, a strand is produced from a number of threads and a braid is produced from a number of strands. The filaments 13 are all oriented in the same direction, here the general direction of the tube 11 shown in FIG. 1 by its axis 9.

The filaments 13 all have the same section, which remains constant over their length.

Here the filaments 13 are solid and have a circular contour.

The wick that constitutes the plug 12 has a maximum rate of filling with filaments when the filaments 13 are contiguous.

There therefore exist between the filaments 13 interstices 20 each delimited by three contiguous filaments 13 and having in section (the interstices 20 extend the whole length of the filaments 13) the overall shape of an equilateral triangle the sides of which are curved with their concave side facing inward.

It can be shown that to be accommodated in an interstice 20 a spherical particle must have a maximum diameter equal to approximately ⅙th of the diameter of the filaments 13.

The particle 21 shown in FIG. 3 has such a diameter.

For example:
to block particles having a diameter greater than or equal to 5 μm the filaments 13 must have a diameter of the order of 30 μm;
with filaments 13 having a diameter of 6 μm, the wick that constitutes the plug 12 blocks particles having a diameter greater than 1 μm (and allows through particles of smaller diameter).

One simple way to determine the number of filaments that must be employed to form the wick that constitutes the plug 12 is to calculate the area of the hexagon for which the distance between the opposite sides corresponds to the diameter of a filament 13. Such hexagons are shown in FIG. 3. The same hexagons are shown in FIGS. 4 to 7.

The area of the wick in section (here the area of the inside section of the tube 11) is then divided by the area of one such hexagon to obtain the number of filaments 13.

For example, approximately 65 000 filaments are required to form the wick that constitutes the plug 12 of a tube 11 having an inside diameter of 1.6 mm with filaments having a diameter of 6 μm.

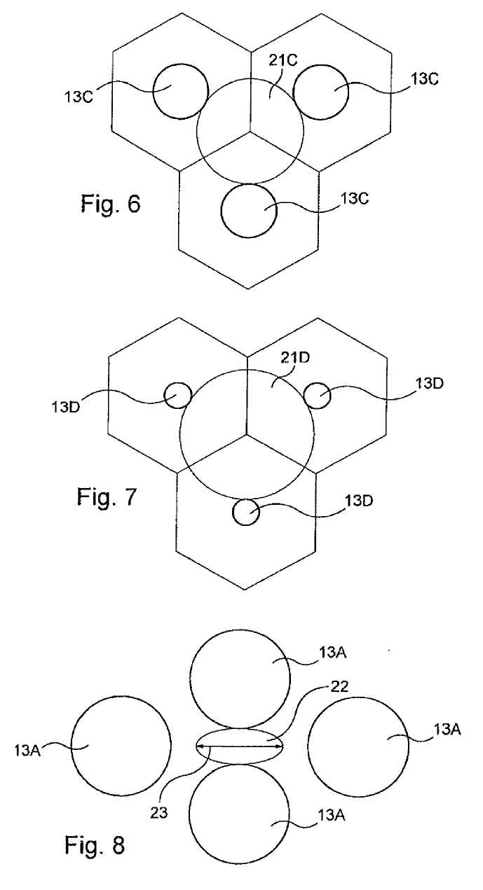

In the variants shown in FIGS. 4 to 7, the number of filaments employed remains the same but the rate of filling with filaments decreases: the diameter of the filaments 13A (FIG. 4) is less than the diameter of the filaments 13, the diameter of the filaments 13B (FIG. 5) is less than the diameter of the filaments 13A, the diameter of the filaments 13C (FIG. 6) is less than the diameter of the filaments 13B, and the diameter of the filaments 13D (FIG. 7) is less than the diameter of the filaments 13C.

The maximum diameter of particles that can be inserted between the filaments varies accordingly, of course.

Thus the particle 21A (FIG. 4) has a larger diameter than the particle 21 and a smaller diameter than the filaments 13A, the particle 21B (FIG. 5) has a larger diameter than the particle 21A and substantially the same diameter as the filaments 13B, the particle 21C (FIG. 6) has a larger diameter than the particle 21B and a larger diameter than the filaments 13C, and the particles 21D (FIG. 7) has a larger diameter than the particle 21C and a larger diameter than the filaments 13D.

FIG. 8 shows the same variant as FIG. 4 but the particle shown is flattened instead of spherical (here it is an ellipsoid).

It will be noted that the greatest dimension 23 of the particle 22 is much greater than the diameter of the particle 21A.

For example, for a diameter of the filaments 13A of the order of 10.7 μm the diameter of the particle 21A is of the order of 5.9 μm and the greatest diameter 23 of the particle 22 is of the order of 8.9 μm.

In some circumstances it is beneficial to consider flattened particles such as the particles 22 rather than spherical particles, notably when the substance to be stored in the straw is semen and the spermatozoa contained in the semen must not be able to penetrate into the wick of filaments. The head of a spermatozoon, which is its widest part, is not spherical but flattened.

It will be noted that in the variants shown in FIGS. 4 to 8 if the filaments are not contiguous in the wick they are considered homogeneously distributed.

Trials have shown that this hypothesis is proven in practice.

When the wick has a maximum filling rate (filaments 13) it is possible to block relatively small particles but the wick then has a low permeability to air.

If the fiber filling rate is particularly low, as with the filaments 13D, the wick is highly permeable to air but only particles of relatively large dimensions can be blocked.

In the variants shown in FIGS. 9, 10 and 11 the filaments are not solid but tubular.

The filaments 13E in the variant shown in FIG. 9 are contiguous and so a particle 21E that can enter the interstices that exist between the filaments has a maximum diameter of the order of ⅙th of the diameter of the filaments 13E.

The diameter of the passage 18 inside each filament 13E is of the same order of magnitude.

Thus the wick formed with the filaments 13E has the same particle blocking capacities as if the filaments 13E were solid but offers improved permeability because the internal passages 14 of the filaments are added to the interstices between filaments.

In the variant shown in FIG. 10 the filaments 13F are also contiguous but the diameter of their internal passages 18F is greater than that of the particles 21F.

It is therefore the diameter of the passages 18F that has to be considered to determine the diameter of the particles that the wick produced with the filaments 13F is capable of blocking.

In the variant shown in FIG. 11 the filaments 13G are not contiguous and it is possible to insert between them a particle 21G whose maximum diameter is greater than the diameter of the internal passages 18G of the filaments 13G.

Thus the minimum diameter of the blocked particles is that of the particle 21G.

Generally speaking, the diameter of the filaments and where applicable of their internal passage and the filament filling rate are chosen as a function of the circumstances of use of the straw.

Depending on the choices made, it is possible to block at the end 16 of the wick such as 12 the material that accounts for the value of the substance introduced into the straw, for example the spermatozoa if it is semen for use in artificial insemination.

As a function of the characteristics of the wick such as 12 and the characteristics of the substance coming up against it, liquid can either flow or not flow through this wick.

It will be noted that if the liquid passes through the wick such as 12 this does not result in any loss of the most valuable material since that material is filtered at the end such as 16 of the wick such as 12.

Another parameter that influences the performance of the plug, notably in the matter of its permeability to air and its friction relative to the tube 11, is the length of the filaments such as 13.

At present, for an animal semen storage straw having an inside diameter of 1.6 mm, the best results seem to be obtained with a plug made of 257 963 solid filaments having a diameter of 3 μm and a length of 5 mm.

In the wick that constitutes such a plug the filaments are contiguous and the wick blocks at its end spherical particles having a diameter greater than 0.46 μm.

The material from which the filaments are made is polyester.

Many other materials can be used to form the filaments such as 13, for example carbon fibers or glass fibers.

It will be noted that polyester, carbon and glass are non-absorbent, with the result that bringing filaments made from these materials into contact with a liquid does not modify their geometrical characteristics.

Absorbent materials such as an alginate can equally be used on condition that modifications of the geometrical characteristics induced by contact with a liquid are taken into account to predetermine the porosity.

If the geometrical characteristic modifications occur over a relatively long time, for example more than one second, and the aim is to avoid any loss of particles in the wick, the geometrical characteristics before absorption of liquid are taken into account.

On the other hand, if the reaction is extremely fast, for example taking of the order of 1/10th of a second, it is possible to take into account the modified geometry.

It will further be noted that other elements can be taken into account to determine the filtration capacities of a wick such as that which constitutes the plug 12, for example local modification of the diameter of the filaments at their ends, likely to be caused by cutting the filaments.

It will finally be noted, with regard to the capacity of a wick such as that which constitutes the plug 12 to block the passage of a liquid or not, that this capacity brings into play the surface tension and thus the physical-chemical characteristics of the filaments and the liquid present in the substance coming up against the filaments.

It is moreover possible to adjust these characteristics with a surface treatment of the filaments, for example a treatment using silicone oil.

Variants of the straw 10 are described next with reference to FIGS. 12 to 17.

For each of these variants, there have been employed for similar elements the same reference numbers increased by 100, 200, 300, 400, 500 and 600, respectively.

The straw 110 shown in FIG. 12 includes a tube 111 identical to the tube 11 and a plug 112 that includes a wick 30 similar to the wick that constitutes the plug except it is surrounded by a sheath 31 disposed between the wick 30 and the tube 111.

The sheath 31 is formed by a braid, silicone or a polymer.

The thickness of the sheath 31 is similar to the thickness of the tube 111.

The straw 210 includes a tube 211 identical to the tube 11 and a plug 212 similar to the plug 112 except that the sheath 32 has a thickness that is several times that of the tube 211 and consequently the wick 33 has a smaller diameter than the wick 30.

The straw 310 shown in FIG. 14 includes a tube 311 identical to the tube 11.

The plug 312 is tripartite: it includes conventional gelling powder 35 sandwiched between a wick 36 similar to the wick 12 and a bung 37 similar to the plug 112, the wick 36 being disposed on the side where the straw 310 is filled with the liquid-based substance (on the side of the end 315 of the tube 311 that is farthest from the plug 312).

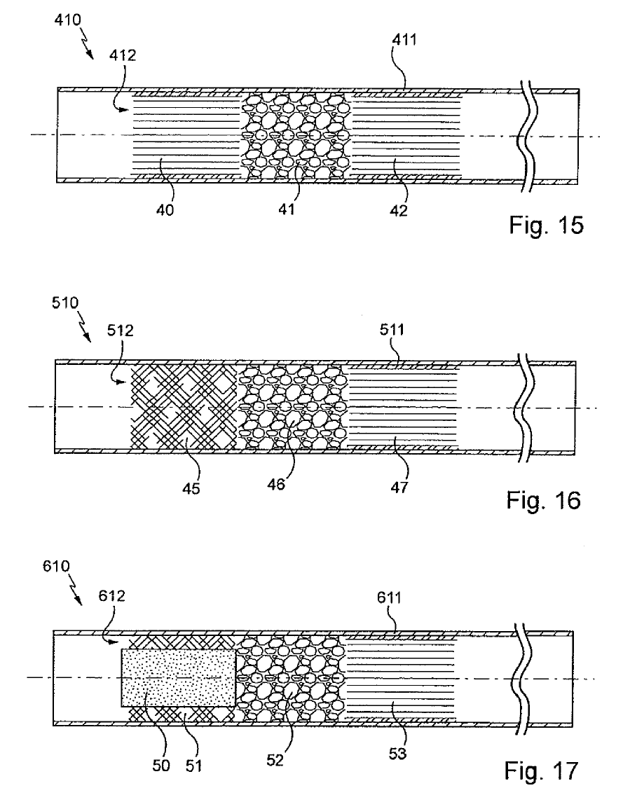

The straw 410 shown in FIG. 15 includes a tube 411 identical to the tube 11 and a plug 412 identical to the plug 312 except that the inner bung 42 is made from a wick surrounded by a sheath. The powder 41 is identical to the powder 35 and the external bung 40 and the internal bung 42 are identical to the plug 112.

The straw 510 shown in FIG. 16 includes a tube 511 identical to the tube 411 and a plug 512 identical to the plug 412 except that the external bung 40 is replaced by a conventional braided bung 45. The powder 46 and the internal bung 47 are identical to the powder 41 and the internal bung 42, respectively.

The straw 610 shown in FIG. 17 includes a tube 611 identical to the tube 11 and a plug 612 identical to the plug 412 or 512 except that the external bung 40 or 45 is replaced by a solid core 50 covered with a braid 51 forming a sheath. The powder 52 and the internal buffer 53 are identical to the powder 41 or 46 and the internal buffer 42 or 47, respectively.

It will be seen that in each of the straws 10, 110, 210, 310, 410, 510 and 610 the plug 12, 112, 212, 312, 412, 512 or 612 includes a wick, one end of which forms the end of the plug facing toward the end of the tube farthest from the plug, such as the end 15 of the tube 11.

Thus this end of each of these wicks is in the area of the plug encountered first by the substance introduced into the straw.

Thus each of these plugs has the advantages disclosed hereinabove in relation to the plug 12, namely preventing the most valuable material, relative to which the wick of filaments has been dimensioned, being able to penetrate into and therefore to be lost in the plug.

In variants that are not shown, the plug of the straw is used with a filtering element different from that described above, for example of microporous material, or produced from elementary units with calibrated dimensions other than filaments.

Numerous other variants are possible as a function of circumstances, and in this regard it is pointed out that the invention is not limited to the examples described and shown.

1. Straw for storing a predetermined dose of liquid-based substance, notably a biological substance, said straw comprising a tube (11; 111; 211;
311; 411; 511; 611) provided with a plug (12; 112; 212; 312; 412; 512; 612), wherein said plug includes a filtering element (12, 112, 212, 36; 42; 47; 53) such that particles exceeding a predetermined dimensional threshold cannot penetrate into it, one end (16) of said filtering element being part of the end of the plug (12; 112; 212; 312; 412; 512; 612) facing toward the end (15; 115) of the tube farthest from the plug.

2. Straw according to claim 1, wherein said filtering element includes a wick (12; 30; 33; 36) of predetermined section formed of a multitude of filaments (13; 13A; 13B; 13C; 13D; 13E; 13F) each oriented in the same general direction (9), each of said filaments having the same predetermined constant section over its length and one end (16) of said wick forming said end of the plug.

3. Straw according to claim 2, wherein said wick (12; 30; 33; 36) has a length at least equal to its radius.

4. Straw according to claim 1, wherein each of said filaments (13; 13A; 13B; 13C; 13D; 13E; 13F) has a rounded contour.

5. Straw according to claim 4, wherein the diameter of said filaments (13; 13A; 13B; 13C; 13D; 13E; 13F) is less than six times a prefixed diameter so that a spherical particle (21; 21A; 21B; 21C; 21D; 21E; 21F; 21G) having a diameter greater than or equal to that prefixed diameter is blocked by said wick.

6. Straw according to claim 1, wherein said filaments (13; 13A; 13B; 13C; 13D) are solid.

7. Straw according to claim 1, wherein said filaments (13E; 13F; 13G) are tubular.

8. Straw according to claim 7, wherein the internal passages (18; 18F; 18G) of said filaments have a diameter less than a prefixed diameter so that a spherical particle (21E; 21F; 21G) having a diameter greater than or equal to that prefixed diameter is blocked by said wick.

9. Straw according to claim 1, wherein said wick (12; 30; 33; 36) has a retaining element (11; 31; 32) around it.

10. Straw according to claim 9, wherein said retaining element is a tube (11) in which said wick is engaged.

11. Straw according to claim 9, wherein said retaining element is an individual retaining sheath (31; 32) of said wick.

12. Straw according to claim 1, wherein said filaments (13; 13A; 13B; 13C; 13D; 13E; 13F) receive a surface treatment.

13. Straw according to claim 12, wherein said surface treatment uses silicone oil.

14. Straw according to claim 1, wherein said plug (12) comprises exclusively said wick.

15. Straw according to claim 13, wherein said plug (112; 212; 312; 412; 512; 612) includes a sheath (31; 32) around said wick (30; 33).

16. Straw according to claim 1, wherein said plug (312; 412; 512; 612) contains gelling powder (35; 41; 46; 52).

17. Straw according to claim 16, wherein said plug (312; 412; 512; 612) includes a bung (37; 40; 45; 50; 51) between said powder (35; 41; 46; 52) and the end of said tube (311; 411; 511; 611) nearest the plug.

18. Straw according to claim 17, wherein said bung (37; 40) includes one of said wicks around which is a sheath.

19. Straw according to claim 17, wherein said bung (45) is formed by a braid of fibrous material.

20. Straw according to claim 17, wherein said bung includes a solid core (50) surrounded by a braid (51) sheathing said core.
